(19) United States
(12) Patent Application Publication
(10) Pub. No.: US 2010/0145199 A1
(43) Pub. Date: Jun. 10, 2010

(54) Cancer Detection System

(76) Inventors: Kristie Johnson, New South Wales (AU); Dennis Wayne Chicken, London (GB); Mohammed Keshtgar, Hampstead (GB); Stephen Glendening Bown, Herts (GB); Hitoshi Iida, Hamamatsu City (JP); Kazuji Matsumoto, Hamamatsu City (JP)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/224,693

(22) PCT Filed: Jan. 3, 2007
(86) PCT No.: PCT/GB2007/000729

§ 371 Date: Oct. 8, 2009

(30) Foreign Application Priority Data
Mar. 2, 2006 (GB) 0604231.1

(51) Int. Cl. A61B 6/00

(52) U.S. Cl. 600/476

(57) ABSTRACT

A cancer detection system (100) comprising a light generator (34) and a probe (24) including first and second light guides (26, 27). The first guide propagates the examination light emitted from the light generator and irradiates tissue (20) with the light. The second guide propagates the light scattered by the tissue, and a spectrum detector (36) measures the spectrum of the scattered light. A decision device (40) determines if cancer exists in the tissue, based on the measured spectrum. A fiber optic plate (22) is provided between the probe and tissue, and a moving mechanism (16) relatively moves the first and second light guides with respect to the tissue. Immersion oil may be disposed between the second end face of the fiber optic plate and the end portions of the first and second light guides.

The present invention relates to a system for detecting cancer in a tissue by measuring the spectrum of light scattered by the tissue.

Sentinel lymph node biopsy is an operation carried out for patients with breast cancer. The sentinel lymph node (SLN) is the first lymph node to receive lymph flow from the breast and hence is the first lymph node that a breast cancer will spread to. If cancer is found in the SLN, axillary lymph node dissection (removal of all the lymph nodes within the armpit) is carried out, and if not, the wound is closed without removing any further lymph nodes. Pathologists can detect cancer in SLNs by examination of the SLNs under a microscope, using the techniques of Frozen Section or Touch Imprint Cytology.

An alternative method for determining whether tissue contains tumour or not using Elastic Scattering Spectroscopy (ESS) is disclosed in Kristie S. Johnson et al., “Elastic scattering spectroscopy for intraoperative determination of sentinel lymph node status in the breast,” Journal of Biomedical Optics, November/December 2004, Vol. 9, No. 6, p. 1122-1128, and Judith R. Mourant et al., “Elastic Scattering Spectroscopy as a Diagnostic Tool for Differentiating Pathologies in the Gastrointestinal Tract Preliminary Testing,” Journal of Biomedical Optics, April 1996, Vol. 1, No. 2, p. 192-199. Furthermore, U.S. Pat. No. 5,303,026 to Strobl et al. discloses a system for spectroscopic analysis of scattering media, and U.S. Pat. No. 6,526,299 to Pickard discloses a method for processing an elastic scattering spectrum taken from human tissue. In the prior art, an examiner puts the tip of a probe onto a tissue, irradiates the tissue with examination light, collect the light scattered by the tissue, and measure the spectrum of the scattered light.

In the prior art, the examiner places the probe on only a few sites on the tissue, and the spectra for many other sites on the tissue are not measured. Since the examiner decides at random where to put the probe, it is likely that the probe will be placed on to portions without cancer and the result of the examination will be incorrect. If the examiner puts the probe on to more portions, it is possible to improve the accuracy of the examination; however, the examination will take much longer.

It is an object of the invention to provide a cancer detection system capable of accurately and rapidly determining if cancer exists anywhere in a tissue.

The cancer detection system in accordance with the present invention comprises: a light generator for emitting examination light; a first light guide for propagating the examination light emitted from the light generator to irradiates a tissue with the examination light; a second light guide for receiving and propagating the examination light scattered by the tissue; and a spectrum detector for detecting the examination light from the second light guide to measure the spectrum of the detected light. The procedure may also be undertaken using a single bidirectional fibre or multiple fibres.

The cancer detection system further comprises: a fibre optic plate having a first end face to face the tissue, a second end face for receiving the examination light from the first light guide and emitting the scattered examination light into the second light guide, and moving mechanism for moving the first and second light guides with respect to the tissue.

A thin, transparent membrane may be provided between the tissue and the fibre optic plate, to improve contact between the two and keep the fibre optic plate clean so it can easily be used on multiple specimens

The fibre optic plate may be configured of multiple optical fibres bundled together, which transmit light entering one of the end faces to the other, with the two dimensional position of the light being maintained.

A decision device for determining if cancer exists in the tissue, based on the measured spectrum, may also be provided.

The cancer detection system may further comprise a stage on which the tissue is to be placed. The moving mechanism may move the first and second light guides with respect to the tissue by moving the stage and the fibre optic plate or by moving the light guides.

Each of the first and second light guides may be an optical fibre having an end face opposing the second end face of the fibre optic plate. The cancer detection system may further comprise a member for holding the portions of the optical fibres including the end faces opposing the second end face.

An immersion liquid may be disposed between the second end face of the fibre optic plate and the end portions of the first and second light guides.

The cancer detection system may further comprise a lens system for transmitting the examination light from the first light guide to optically couple the examination light with the second end face of the fibre optic plate, and for transmitting the examination light scattered by the tissue and emerging from the second end face of the fibre optic plate to optically couple the scattered examination light with the second light guide.

The cancer detection system may further comprise a mirror system for reflecting the examination light from the first light guide to optically couple the examination light with the second end face of the fibre optic plate, and for reflecting the examination light scattered by the tissue and emerging from the second end face of the fibre optic plate to optically couple the scattered examination light with the second light guide.

The first light guide may have a first end portion in which a plurality of optical fibres are bundled, and a sheet-shaped second end portion in which the plurality of optical fibres are arranged in a plane. The second light guide may have sheet-shaped end portions having a plurality of optical fibres arranged in a plane. In the first light guide, the first end portion may receive the examination light from the light generator, and the second end portion may emit the examination light to the second end face of the fibre optic plate. In the second light guide, one of the sheet-shaped end portions may receive the scattered examination light from the second end face of the fibre optic plate, and the other of the sheet-shaped end portions may transmit the scattered examination light to the spectrum detector.

The light generator may generate light having a predetermined range of wavelengths as the examination light. The spectrum detector may include a photodetector for measuring the intensities of a plurality of components of the scattered examination light. The components may have a plurality of wavelength ranges, each of which is narrower than the predetermined wavelength range.

The light generator may generate light in a plurality of wavelength ranges. The light generator may be able to switch from one wavelength range to another. The spectrum detector may include a photodetector for measuring the intensity of the scattered examination light. The cancer detection system may further comprise a controller for causing the light generator to switch the wavelength range of the examination light from one wavelength range to another and for causing the photodetector to measure the intensity of the examination light in each of the wavelength ranges. The system may also be used with other optical diagnostic techniques including, but not limited to, Raman spectroscopy and fluorescence.

The present invention will be more fully understood from the following detailed description and the accompanying drawings. The accompanying drawings are only illustrative and are not intended to limit the scope of the present invention.

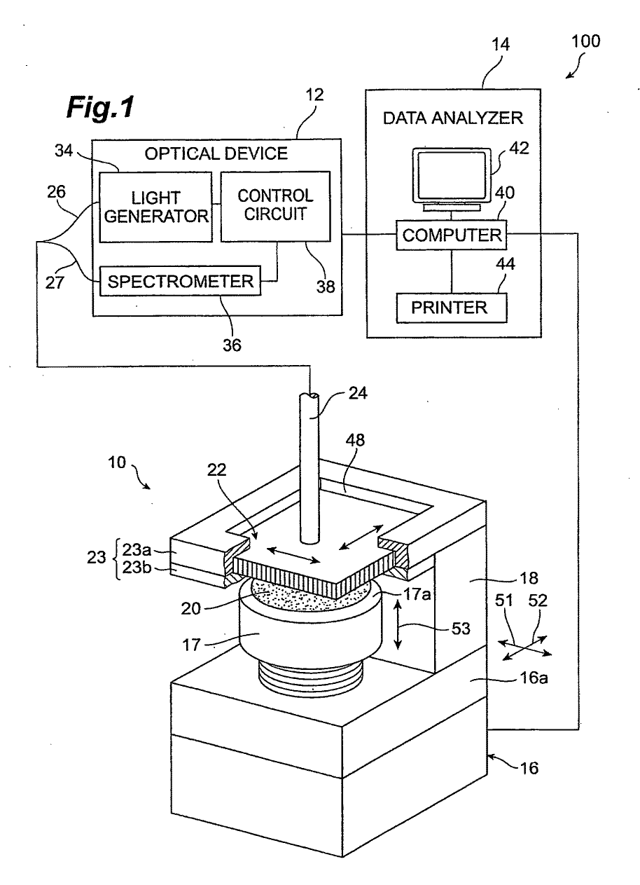

FIG. 1 is a schematic view showing a cancer detection system in accordance with the first embodiment.

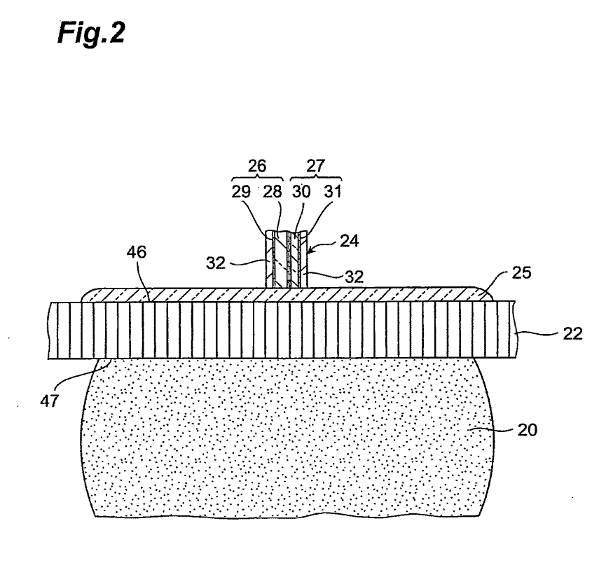

FIG. 2 is an exploded partial sectional view showing a fibre optic plate and a probe.

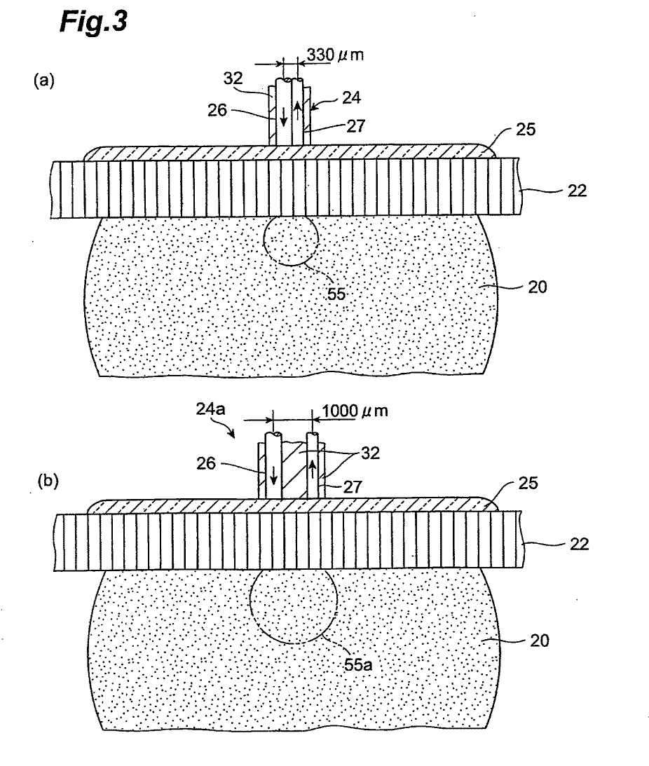

FIG. 3 is a partial sectional view showing change in measurement volume corresponding to the intervals between fibres.

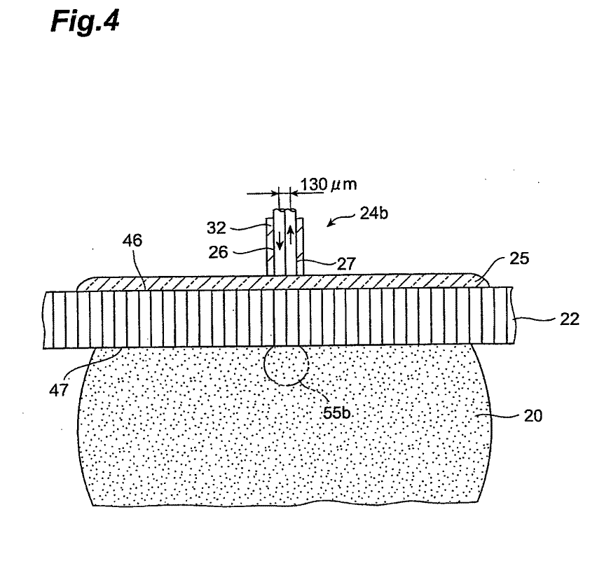

FIG. 4 is a partial sectional view showing a region that can be measured if the diameters of the fibres are small.

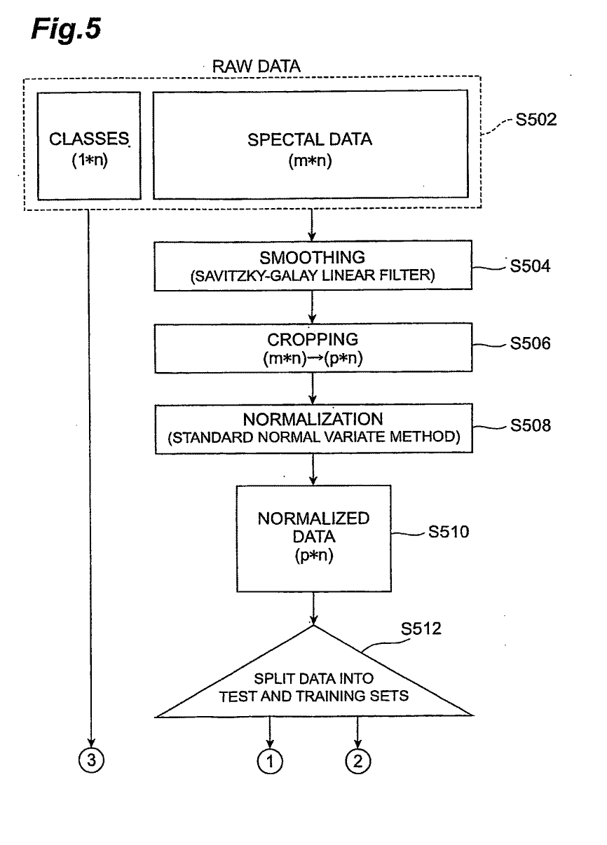

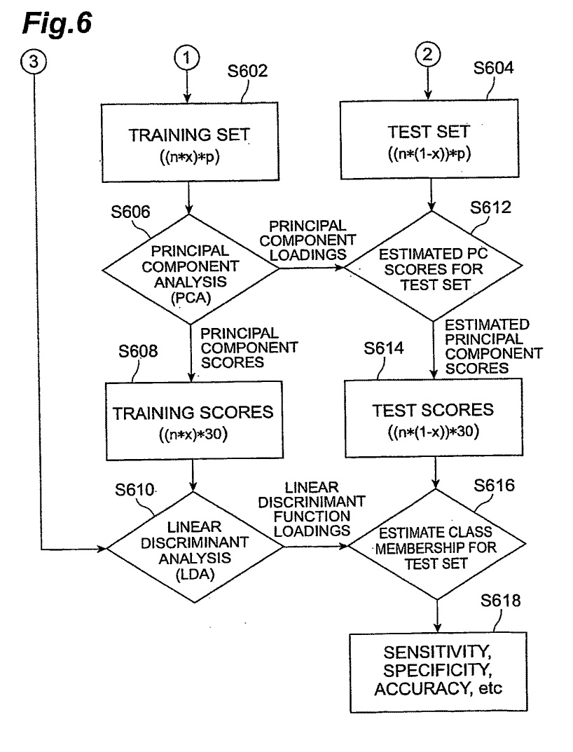

FIGS. 5 and 6 are flowcharts showing how to establish a mathematical model for the cancer examination.

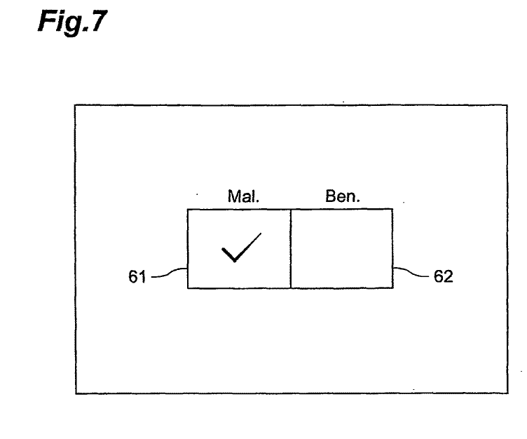

FIGS. 7 to 10 are views showing examples of how to display the result of the examination.

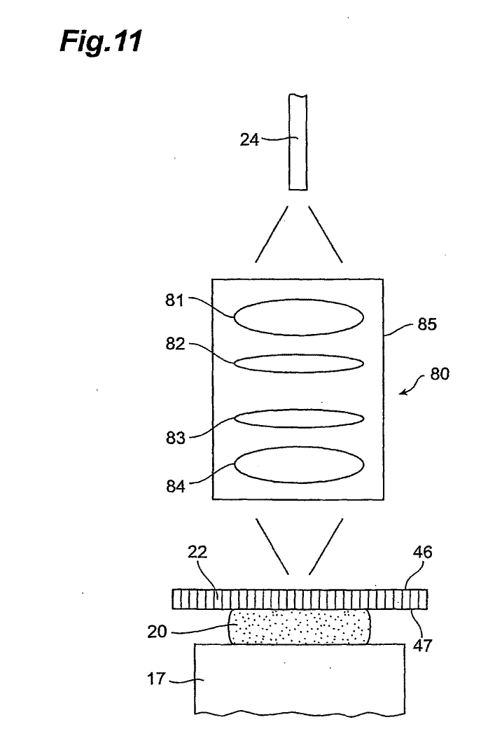

FIG. 11 is a schematic view showing the optical coupling between a fibre optic plate and a probe in the second embodiment.

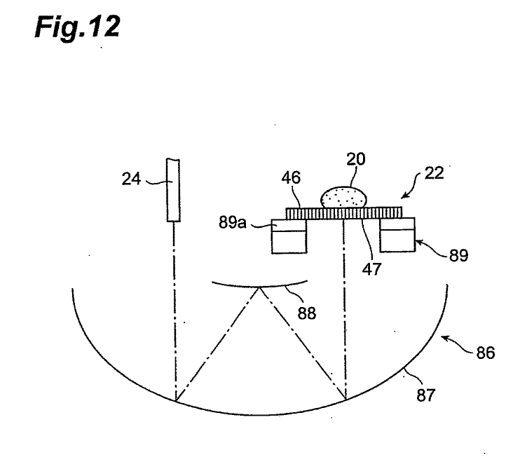

FIG. 12 is a schematic view showing the optical coupling between a fibre optic plate and a probe in the third embodiment.

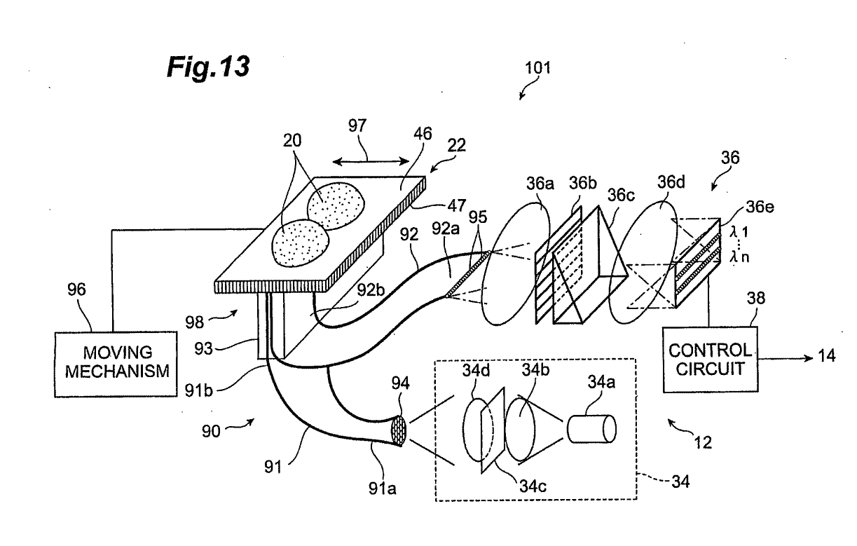

FIG. 13 is a schematic view showing a cancer examination system in accordance with the fourth embodiment.

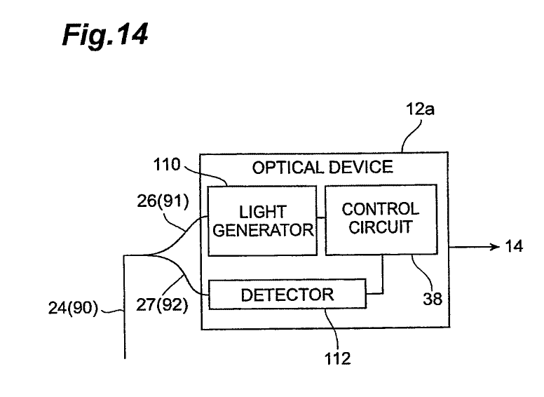

FIG. 14 is a schematic view showing an optical device for a pre-dispersion cancer examination system.

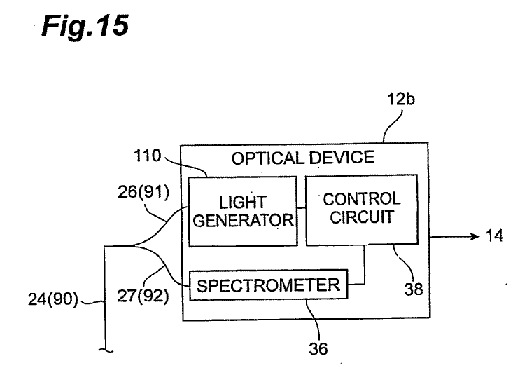

FIG. 15 is a schematic view showing an optical device for a double-dispersion cancer examination system.

The preferred embodiments of the present invention will be described below in greater detail with reference to the accompanying drawings. To facilitate understanding, identical reference numerals are used, where possible, to designate identical or equivalent elements that are common to the embodiments, and, in subsequent embodiments, these elements will not be further explained.

### FIRST EMBODIMENT

FIG. 1 is a schematic view showing a cancer detection system in accordance with the present embodiment. The system 100 has a scanner 10, an optical device 12, and a data analyzer 14, and performs elastic scattering spectroscopy (ESS) to examine a tissue 20.

The scanner 10 includes a movable stage 16, a fibre optic plate (FOP) 22, and a probe 24. The movable stage 16 has a top plate 16a capable of moving in directions 51 and 52 as shown in FIG. 1. The top plate 16a is provided with a sample stage 17 capable of moving up and down in a direction 53. On the sample stage 17, a tissue 20 to be examined is placed. The top plate 16a of the movable stage 16 has a support 18 thereon, and a frame 23 is attached to the top of the support 18 in order to fix the FOP 22 to the support 18. The frame 23 holds the edge of the FOP 22. The frame 23 is configured of a top plate 23a and a bottom plate 23b. The top plate 23a has a groove into which the FOP 22 is fitted. When examining the tissue 20, the height of the sample stage 17 is adjusted so that FOP 22 touches the tissue 20 on the sample stage 17.

FIG. 2 is an exploded partial sectional view showing the FOP 22 and the probe 24. The FOP 22 is shaped in a parallel plate, and has two opposite end faces, i.e., a top face 46 and a bottom face 47. The FOP 22 is configured of multiple optical fibres bundled together, and transmit light entering one of the end faces to the other, with the two dimensional position of the light being maintained. In this embodiment, the numeral aperture (NA) of the FOP is 1.0.

The probe 24 is disposed so that one of the ends thereof faces the top face 46 of the FOP 22. The probe 24 has an optical fibre (hereafter referred to as the “irradiating fiber”) 26 for propagating examination light and irradiating the tissue 20 with the propagated examination light, an optical fibre (“collecting fibre” hereinbelow) 27 for collecting the examination light scattered by the tissue 20. The irradiating fibre 26 includes a core 28 for confining and transmitting the examination light, and a cladding 29 that covers the side face of the core 28. Likewise, the collecting fibre 27 includes a core 30 for confining and transmitting the scattered examination light, and a cladding 31 that covers the side face of the core 30. In this embodiment, the diameter of the core 28 is 400 μm, and that of the core 30 is 200 μm. The side faces of these optical fibres are covered by coating 32 so as to hold the fibres together.

In order to prevent the examination light emitted from the irradiating fiber 26 from being reflected by the top face 46 to enter the collecting fiber 27, immersion oil 25 is interposed between one of the end faces of the probe 24 and the top face 46 of the FOP 22. The end of the probe 24 is preferably apart from the FOP 22 by a slight distance (e.g., approximately 0.1 mm-0.5 mm) because it is necessary to slide the probe 24 relatively easily with respect to the FOP 22 to scan the tissue 20. If there were air between the FOP 22 and probe 24, undesired light that has not passed through the tissue 20 would be likely to be incident on the collecting fibre 27. To prevent this occurrence, the immersion oil 25 is applied to the surface of the FOP 22 so that the end of the probe 24 slides on the immersion oil 25. Consequently, it is possible to prevent the reflection of the examination light by the top face 46 of the FOP 22, and the examination light is allowed to travel between the FOP 22 and probe 24 without the beam diverging.

The immersion oil 25 has a refractive index at least higher than that of air, i.e., 1. More preferably, the immersion oil 25 has the same refractive index as at least one of the cores of the FOP 22, irradiating fibre 26 and collecting fibre 27.

It is possible to adjust the volume of the region that can be measured in the tissue 20 by changing the distance between the irradiating fibre 26 and the collecting fibre 27. FIG. 3 is a partial sectional view showing the measurable volume corresponding to the interval between these fibres. As shown in FIG. 3(a), the irradiating fibre 26 is immediately adjacent to the collecting fibre 27, and the interval between these fibres (that is, the distance between the centers of the fibres) is 330 μm. In this case, the examination light is allowed to enter the region 55 from the irradiating fibre 26, and the examination light scattered from the region 55 can be collected into the collecting fibre 27. In contrast, when a probe 24a with the wider distance, such as 1000 μm, between the irradiating and collecting fibres 26 and 27, as shown in FIG. 3(b), the examination light is allowed to enter the region 55a with a larger volume, and the examination light scattered from the larger region 55a can be collected.

As shown in FIG. 4, decreasing the diameters of the irradiating and collecting fibres 26 and 27 enables a measurable region 55b with a small volume to be obtained, and therefore it is possible to acquire spectral information at higher resolution.

Referring to FIG. 1 again, the optical device 12 has a light generator 34, spectrometer 36 and control circuit 38. The light generator 34 is a device for generating the examination light with which the tissue 20 is irradiated, and includes a light source emitting light with a sufficiently wide range of wavelengths. Examples of the light source are a Xenon (Xe) lamp, Xe flash lamp, halogen lamp and LED (e.g., white LED). The white LED may be configured of an LED that emits light in the blue or ultraviolet region, and a fluorescent material to be excited by the emitted light. Alternatively, the light source may include a plurality of LEDs, mix the light from these LEDs, and emit the mixed light.

In this embodiment, a Xe lamp is used as the light source. The spectrum of the light emitted from the Xe lamp has a peak rising at a wavelength near 200 nm as well as a peak in the near-infrared region. In this embodiment, an optical filter for transmitting the light from the Xe lamp is also provided in the light generator 34 to generate the examination light having a spectrum that is smooth in a range between 300 nm and 1100 nm. As examples, suitable optical filters are Filters #3308 and #2 from the Posco corp.

The white LED which may be used as the light source instead of the Xe lamp emits light with a relatively narrow range of wavelengths, i.e., about 380 nm-about 780 nm. On the other hand, the halogen lamp emits light with a low intensity in the ultraviolet region but with stronger emission in the near infrared region. When using the halogen lamp, an optical filter may be provided in the light generator 34 to generate the examination light having a flat spectrum. Alternatively, the system can be calibrated using a reference white surface such as Spectralon.

One of the ends of the irradiating fibre 26, which end is on the side away from the FOP 22, is optically coupled with the light generator 34, so that the examination light generated by the light generator 34 is introduced into the irradiating fiber 26. The examination light is propagated to the other end of the irradiating fibre 26 and transmitted through the immersion oil 25 and the FOP 22 to irradiate the tissue 20. The examination light is repeatedly scattered and absorbed in the tissue 20. Part of the scattered examination light passes through the FOP 22 to enter one of the ends of the collecting fibre 27. To the other end of the collecting fibre 27, a spectrometer 36 is optically coupled. The collecting fibre 27 propagates and introduces the examination light scattered by the tissue 20 into the spectrometer 36.

The spectrometer 36 has a diffraction element such as a concave grating, and a line sensor for receiving light emerging from the diffraction element. The diffraction element receives the examination light from the collecting fibre 27 and disperses the examination light in different directions depending on the wavelengths of the examination light. The line sensor is positioned so that the dispersed beams are incident on the elongated input face of the line sensor. The line sensor includes photoelectric converters arranged along the length of the sensor. The photoelectric transducers measure the intensities of the examination light at the respective spectroscopic wavelengths which are contained in the wavelength range of the examination light. The spectrometer 36 generates a spectroscopic data signal representing the measured intensities, and sends the data signal to the control circuit 38.

The control circuit 38 includes an A/D converter for receiving and digitalizing the spectroscopic data signal from the spectrometer 36. The control circuit 38 additionally includes a microprocessor that performs necessary preprocessing, such as noise reduction, normalization of the spectral height, and so forth, of the digitalized spectroscopic data signal. The preprocessed spectroscopic data signal is sent to the data analyzer 14. Also the control circuit 38 controls the operations of the light generator 34 and the spectrometer 36 under control of the data analyzer 14.

The sensors will in general be able to measure a maximum intensity. For example, the sensor may output a 12-bit value from 0 to 4095 in which case the maximum intensity is the intensity represented by 4095. In order to avoid saturated spectra in which the peaks of the measured spectra are clipped by the maximum measured intensity of the sensors, the control circuit 38 preferably carries out an autoranging function to reduce or eliminate such clipping.

The data analyzer 14 includes a personal computer 40. The computer 40 analyzes the preprocessed spectroscopic data signal to determine if the tissue 20 contains cancer, as well as controls the operations of the optical device 12 and the movable stage 16 using the control circuit 38. For example, the computer 40 causes the light generator 34 to emit the examination light while causing the movable stage 16 to move the upper plate 16a, thereby detecting the examination light scattered by the tissue 20 using the spectrometer 36 to acquire the spectroscopic data. Thus it is possible to scan the tissue 20 in two dimensions and to obtain the spectroscopic data from all points on the surface of the tissue 20.

To the computer 40, a display device 42 and a printer 44 are connected. The display device 42 displays the result of cancer detection by the system 100. The printer 44 prints the examination result on a paper.

The computer 40 determines if there is cancer in the tissue 20 by analyzing, by any known method, the spectroscopic data resulting from the two-dimensional scan of the tissue 20. In this embodiment, for determining the presence of cancer, the spectroscopic data is evaluated according to a mathematical method previously established. The mathematical model is established by a known method using the principal component analysis (PCA) and the linear discriminant analysis (LDA). In the following, how to establish the mathematical model will be explained with respect to FIGS. 5 and 6, which are flowcharts showing a process carried out to establish the mathematical model.

First, spectral data and class data, which are raw data, are prepared (Step S502).

The spectral data includes data of spectra of the scattered examination light acquired from tissues. Each spectral data includes intensities of a spectrum at spectroscopic wavelengths contained in the wavelength range of the examination light. The spectral data may be acquired using the system 100 or other systems. In general, the examination light is applied at a plurality of sampling points on every tissue, and the spectral data from every sampling point is acquired. In FIG. 5, the number of the spectroscopic wavelengths, that is, the number of the spectral intensities is represented as “m,” and the total number of the spectra acquired from the tissues as “n.”

The class data are data indicating the result of diagnosis, by a pathologist, of the tissues from which the spectral data are acquired, that is, data indicating whether each point in the tissue from which a spectrum is taken is positive or negative for the presence of cancer. The class data are input to the computer 40 and stored.

The raw spectral data are preprocessed in various ways. If the raw spectral data are acquired using the system 100, the control circuit 38 preprocesses the raw spectral data and sends the preprocessed data to the computer 40. On the other hand, if the raw spectral data is acquired using other systems, the preprocessed spectral data is sent to the computer 40 from the outside of the system 100.

More specifically, the raw spectral data is smoothed using the Savizky-Golay linear filter (Step S504). The Savizky-Golay Linear Filter is described in Savitzky et al., “Smoothing and Differentiation of Data by Simplified Least Squares Procedures,” ANALYTICAL CHEMISTRY, Vol. 36, No. 8, 1964, p. 1627-1639.

Then, the smoothed spectral data are cropped to remove the spectral data for long and short wavelength regions (Step S506). This is for the sake of removing data with a low signal to noise ratio. The cropping reduces the number of spectral intensities in one spectral data from “m” to “p.”

Then the cropped spectral data is normalized by the standard normal variate method (Step S508). As a result, the means of the spectra are set to zero and the standard deviations of the spectra acquired from the sampling points are equalized, and so the normalized spectral data are obtained (Step S510).

The computer 40 splits the normalized spectral data into a training set and a test set (Step S512). The training set is used to calculate a function for determining whether or nor there is cancer in the tissue. The test set is used to examine the accuracy of the calculated determining function. 100% of the training set consists of tissues that are either totally normal or totally cancerous, and the test set includes nodes which may be normal or partially or completely replaced by cancer.

The computer 40 stores the training and test sets in the respective locations in the storage of the computer 40 (Steps S602 and S604). In FIG. 6, “x” represents the ratio of the number of spectra sorted into the training set to the total number “n” of the spectra.

Then, the computer 40 carries out the principal component analysis (PCA) using the training set to determine principal component spectra and calculates principal component scores of the principal component spectra (Step S606). The PCA is one of known multivariate analysis techniques, which introduces new variables, that is, the principal component spectra, and removes overlapping information between the spectra in the training set to reduce the number of variables without reducing the total amount of information. According to the PCA, the multiple spectra in the training set can be evaluated using fewer principal component spectra. The principal component scores represent the degrees to which the respective principal component spectra contribute to a spectrum in the training set. In this embodiment, the number of the principal component spectra is 30.

The principal component scores are defined using principal component loadings determined for the respective spectroscopic wavelengths. The computer 40 is able to calculate the principal component score of each spectrum using the principal component loadings. The computer 40 stores the principal component loadings for each principal component spectrum in the storage. Furthermore, the computer 40 stores a group of principal component scores calculated for each spectrum in the training set in the storage as training scores (Step S608).

Then, the computer 40 reads the class data representing the diagnosis result by the pathologist from the storage, and carries out the linear discriminant analysis (LDA) using the read class data in combination with the training scores (Step S610). The LDA is one of multivariate analysis techniques, and carried out to improve the discrimination between normal tissues and tissues containing cancer. More specifically, a linear discriminant function is calculated from the training scores, which function discriminates between the spectra taken from the tissues diagnosed as negative for the presence of cancer and those taken from the tissues diagnosed as positive by the pathologist. The variables of the linear discriminant function are the principal component scores of the spectra, and the discriminant function is represented as a weighted linear sum of the variables multiplied by the respective factors (weights). These factors are called linear discriminant function loadings. The LDA determines the linear discriminant function by calculating the loading for each principal component spectrum. These loadings are stored in the storage of the computer 40.

Then, it can be determined whether the tissues from which the spectra in the test set are taken are positive or negative, in order to examine the accuracy of the linear discriminant function. First, the computer 40 estimates the principal component scores for the test set using the principal component loadings obtained in Step S606 (Step S612). The estimated principal component scores are stored in the storage as test scores (Step S614). The computer 40 calculates the values of the linear discriminant function, i.e., canonical scores, using the linear discriminant function loadings calculated in Step S610 and the test scores, and estimates the class membership for the test set according to the calculated scores (Step S616). More specifically, the canonical score is calculated from each spectrum in the test set, the calculated canonical score is compared with the predetermined discrimination threshold, and the sampling point in a tissue from which the spectrum is acquired is determined as positive when the canonical score is greater than the discrimination threshold, and negative when not greater than the threshold.

Thereafter, the computer 40 compares the result obtained by the system 100 with the result of the diagnosis by the pathologist, which is stored as the class data, to determine evaluating parameters such as sensitivity, specificity, and accuracy (Step S618). The sensitivity is represented as TP/(TP+FN), and the specificity as TN/(TN+FP), where TP (True Positive) is the number of the sampling points determined as positive by both the system 100 and pathologist, TN (True Negative) the number of the sampling points determined as negative by both the system 100 and pathologist, FP (False Positive) the number of the sampling points determined as positive by the system 100 but negative by the pathologist, FN (False Negative) the number of the sampling points determined as negative by the system 100 but positive by the pathologist.

When these evaluating parameters are appropriate, a set of the linear discrimination function loadings calculated in Step S610 is stored in the storage, and used in the actual cancer examination. The user may instruct the computer 40 to recalculate the linear discriminant function so as to obtain more appropriate evaluating parameters.

In the actual cancer detection, the computer 40 controls the movable stage 16 and the light generator 34 so that the light generator 34 emits the examination light at the predetermined timings while the movable stage 16 moves the tissue 20 and the FOP 22 in at least one of the directions 51 and 52. Thus it is possible to move the probe 24 with respect to the tissue 20 and scan the tissue 20. The examination light passes through the irradiating fiber 26 and the FOP 22 to be applied to the tissue 20, and the examination light scattered by the tissue 20 is collected into the collecting fibre 27. The spectrometer 36 receives the scattered examination light from the collecting fibre 27 and measures the spectrum thereof. Thus it is possible to rapidly acquire the spectra from all appropriate sites on the tissue 20. The control circuit 38 receives the data of the measured spectra from the spectrometer 36 and pre-processes these spectral data as shown in Steps S504-S508. The computer 40 carries out the processes of Steps S612-S616 to determine if cancer is present at each sampling point or not.

Thereafter, the computer 40 displays the result of the examination on the display device 42. The display may be performed in various ways. FIGS. 7-10 show display examples of the examination result.

In the display example shown in FIG. 7, two square frames 61 and 62 are displayed. The frame 61 indicates that tissue 20 is malignant, that is, cancer has been found in the tissue 20, and the frame 62 indicates that the tissue 20 is benign, that is, cancer has not been found in the tissue 20. A checkmark is drawn in either of the frames corresponding to the examination result. For example, when at least one of the sampling points on the tissue 20 is determined as cancerous, the checkmark is drawn in the frame 61 indicating malignancy.

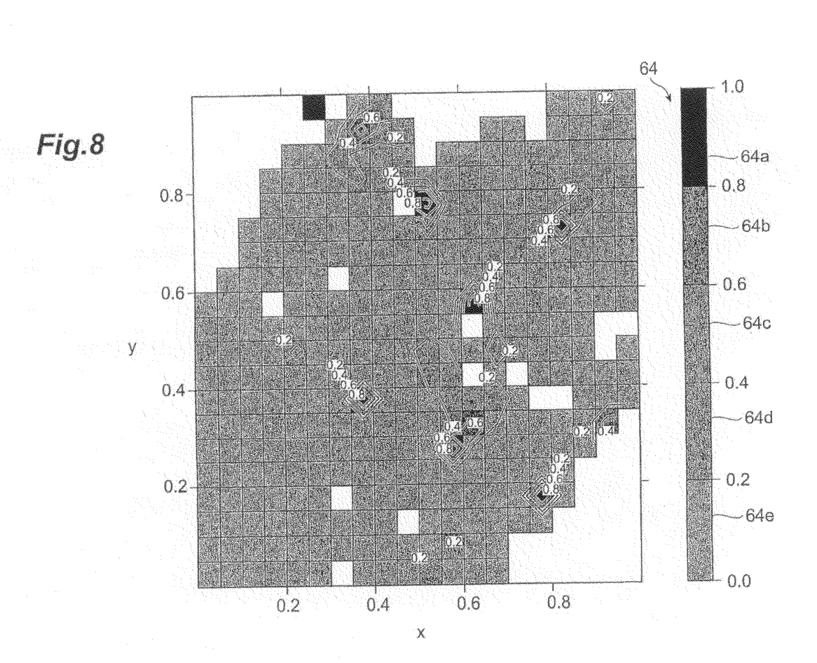

In the display example shown in FIG. 8, the distribution of probability of cancer being present in the tissue 20 is depicted in two dimensions. In the figure, “0.0” means that the existing probability of cancer is zero, that is, the tissue 20 is a normal tissue at a probability of 100%. “1.0” means that the existing probability of cancer is 100%. The black lines are contour lines representing positions having the same existing probability of cancer. Portions in which the contour lines do not finish are those in which the sample data does not exist.

The distribution of the probability of cancer being present is represented by different colours. In a colour bar 64 on the right side of the screen, cells 64a-Me corresponding to various probability ranges are arranged in a line, and coloured with different colours. For example, supposing that the existing probability is represented as P, red indicates 0.8<P≦1.0, reddish purple 0.6<P≦0.8, purple 0.4<P≦0.6, bluish purple 0.2<P≦0.4, blue 0<P≦0.2. Of course, other hues may be used to represent the probability distribution. For example, familiar hues such as orange, yellow, green, and so on may be placed between red and blue. In FIG. 8, regions without hatching are coloured with gray. These regions represent those in which there is no tissue 20 or those in which the tissue 20 did not touch the FOP 22, so an appropriate spectrum could not be recorded.

Although not being adopted in FIG. 8, the examination result may be displayed just using the contour lines. Also, regions between the contour lines may be coloured so as to represent the probability distribution more clearly. For example, a region between a contour line and another one may be coloured with a colour depending on the probability that either of the contour lines indicates. Alternatively, the distribution of the probability of cancer being present may be represented in three dimensions by depicting the probability on the Z-axis.

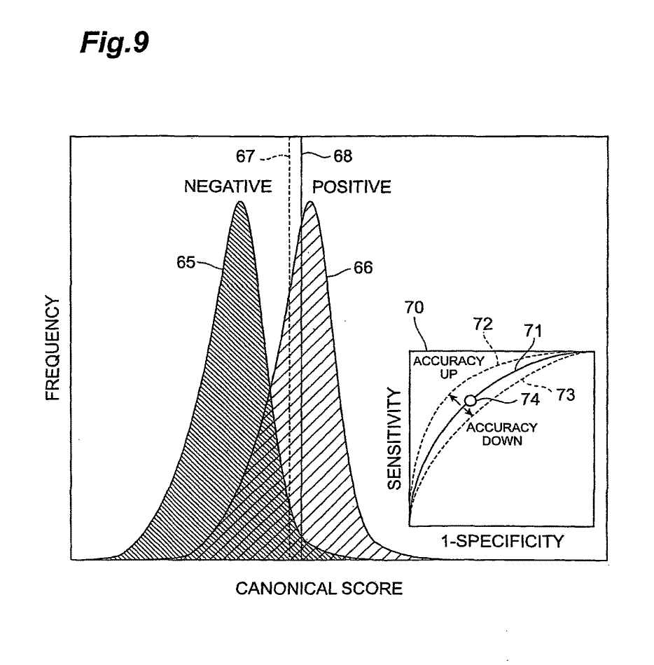

In the display example shown in FIG. 9, in addition to the examination result, the process to determine the linear discriminant function from the training set is displayed. The horizontal axis represents the canonical score calculated from the spectroscopic data of tissue 20. The canonical score indicates how likely the tissue 20 is to be positive. The canonical score ranges from −4 to +4, for example; however, it may be in any range, and is not limited to the range from −4 to +4. The vertical axis represents the frequency at which the canonical score is calculated from the training set. Graphs 65 and 66 indicate the distribution of the canonical scores for the sampling points in the training set that the pathologist has diagnosed as negative and positive, respectively. These graphs may be coloured with different colours. Broken line 67 represents the discrimination threshold used to discriminate between negative and positive results. The canonical score resulting from actually examining the tissue 20 is represented as solid line 68 parallel to the vertical axis.

According to this display example, the user can find the accuracy of the examination result. For example, FIG. 9 shows that solid line 68 representing the examination result is close to the top of the positive distribution 66, but the line 68 also overlaps with the bottom of the negative distribution 65. Thus it is understood that the examination result does not indicate with 100% confidence which sites are positive. In the display example of FIG. 8, how likely the tissue is to be positive at each point being tested is represented as a numeric value, i.e., probability, for example. On the other hand, since the display example of FIG. 9 visually represents the positional relationship between the positive and negative distributions and the canonical score resulting from the examination, the user can find how accurate the diagnosis of the presence or absence of cancer is likely to be.

In window 70 on the lower right of FIG. 9, curve 71 is depicted to indicate the accuracy of the mathematical model produced from the training set, that is, the linear discriminant function. If curve 71 is changed into broken line 72, the accuracy increases, and if curve 71 is changed into broken line 73, the accuracy decreases. Circular mark 74 on curve 71 indicates the discrimination threshold for the canonical score. The specificity and sensitivity are determined depending on the position of circular mark 74. The specificity and sensitivity are not determined uniquely for the linear discriminant function: when the sensitivity increases, the specificity decreases, and when the specificity increases, the sensitivity decreases. When determining the position of circular mark 74, and thereby determining the specificity and sensitivity, the discrimination threshold corresponding to the position of circular mark 74 is displayed as broken line 67.

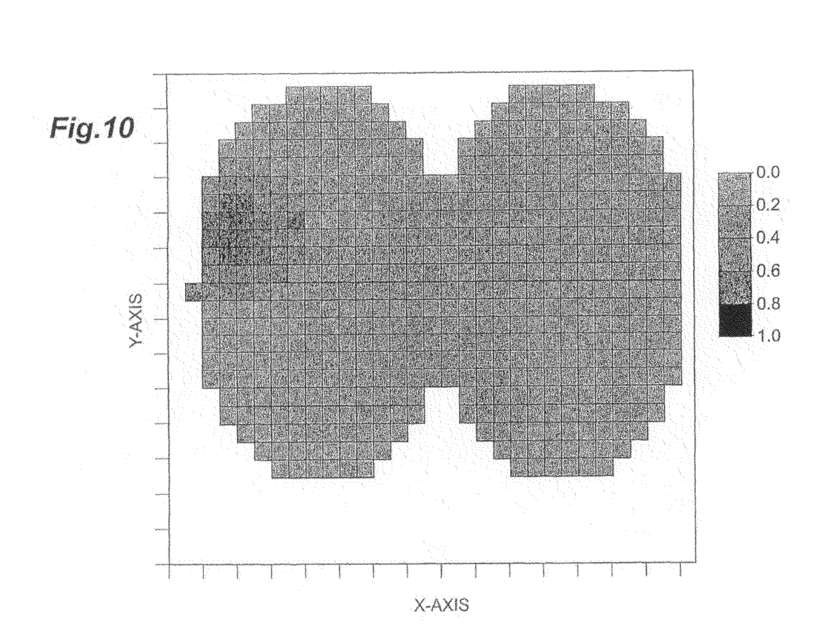

An example will now be explained in which a sentinel lymph node (SLN) from a patient with breast cancer is examined using the cancer detection system 100 to determine if the cancer has metastasized into the SLN. The display example of FIG. 10 shows the result of actually examining a sentinel lymph node, as tissue 20, cut in half. The outline of the cut surface of the tissue 20 is displayed, and the region inside the outline is divided into zones with different colours depending on the probability of cancer existing in those zones. The user can find the cancer metastasis from the presence of the colour indicating high probability of cancer. According to FIG. 10, it is understood that the cancer has metastasized into the tissue 20, and the metastasis is localised to a portion of the tissue.

Sentinel lymph nodes are firm and have irregularities on their surfaces. Therefore, if the probe 24 were to directly touch a sentinel lymph node, it would be impossible to move the probe 24 smoothly with respect to the node. However, in the cancer detection system 100, the FOP 22 is interposed between the probe 24 and the tissue 20 to prevent the probe 24 from directly touching the tissue 20, and therefore it is possible to move the probe 24 with respect to the tissue 20 smoothly. Since the FOP 22 maintains the two-dimensional position of the examination light, it is also possible to obtain sufficient resolution. Consequently it is possible to adequately and rapidly scan the surface of the tissue 20 in two dimensions to acquire the spectra of the elastically scattered light from the whole surface of the tissue. Thus the accuracy of the examination can be improved in comparison with a cancer detection system adapted to acquire the spectra only from a limited number of positions.

The FOP 22 provides an additional effect as follows. If the tip of the probe is placed directly onto tissue without using an FOP, the result of the examination depends significantly on the local pressure applied to the tissue from the probe, particularly if the probe is thin. In contrast, in the cancer detection system 100, the FOP 22 is interposed between the tissue 20 and probe 24, so that local pressure is not applied to the tissue 20, and therefore any variation in the result of the examination depending on the examiners and the examination conditions is reduced.

Using the FOP 22 having a sufficiently large NA (numerical aperture), such as 1.0, enables the divergence of the examination light to be equal to that in the case without using the FOP 22. For example, when using the irradiating fibre 26 having a NA of 0.22, the divergence angle of the examination light is 25.41 degrees regardless of use of the FOP 22 or lack of it. Therefore, even when the FOP 22 is disposed between the tissue 20 and probe 24, the examination result is obtained which is almost the same as that obtained without using the FOP 22. Accordingly, it is possible to carry out data conversion between data obtained using an FOP and data obtained without using an FOP, according to a simple converting expression. Thus, in the cancer detection system 100, spectroscopic information obtained from a single point without using an FOP can also be used as fundamental data (i.e., a training set and a test set). A simple way to correlate measurements with and without the FOP is to calibrate the system to the white reference material, Spectralon with and without the FOP.

### SECOND EMBODIMENT

FIG. 11 is a schematic view showing optical coupling between the FOP 22 and probe 24 in the second embodiment. In this embodiment, the probe 24 is optically coupled with the FOP 22 via a lens system 80. The lens system 80 includes lenses 81, 82, 83 and 84 contained in a lens barrel 85. However, the lens system 80 may be a single lens.

The lens system 80 is positioned so as to form an image of the examination light emerging from the irradiating fibre 26 on the top face 46 of the FOP 22. and also to form an image of the light scattered back from the tissue on the collection fibre 27. Such an arrangement is maintained in spite of the movement of the FOP 22 by the movable stage 16. When the movable stage 16 moves the FOP 22 in two dimensions, the lens system 80 and the probe 24 stay stationary so that they scan across the FOP 22.

In this embodiment, the probe 24 can be positioned sufficiently apart from the FOP 22, and therefore it is not likely for the collecting fibre 27 to receive the examination light reflected by the FOP 22. Thus there is no need to apply the immersion oil 25 to the surface of the FOP 22, thereby providing greater convenience.

### THIRD EMBODIMENT

FIG. 12 is a schematic view showing optical coupling between the FOP 22 and probe 24 in the third embodiment. In this embodiment, the probe 24 is optically coupled with the FOP 22 via a mirror system 86. The mirror system 86 includes a first mirror 87 and a second mirror 88. The first mirror 87 is a concave mirror, and the second mirror 88 is a plane mirror or convex mirror. The examination light is reflected by the mirrors 87 and 88 in turns, and then reflected again by the mirror 87 so as to be transmitted between the FOP 22 and probe 24.

In this embodiment, tissue 20 is placed on the top face 46 of the FOP 22. The FOP 22 is disposed on a top plate 89a of a movable stage 89. The movable stage 89 is able to move the top plate 89a in two dimensions, perpendicularly to the optical axis of the examination light passing through the FOP 22.

The mirror system 86 is positioned so as to form an image of the examination light emerging from the irradiating fibre 26 on the bottom face 47 of the FOP 22. Such an arrangement is maintained in spite of the movement of the FOP 22 by the movable stage 89.

In this embodiment, the probe 24 can be positioned sufficiently apart from the FOP 22, and therefore it is not likely for the collecting fibre 27 to receive the examination light reflected by the FOP 22. Thus there is no need to apply the immersion oil 25 to the surface of the FOP 22, and therefore convenience is improved. A mirror system can also be used to scan across the FOP so it is not necessary to move the FOP to examine an entire section of a lymph node.

### FOURTH EMBODIMENT

FIG. 13 is a schematic view showing a cancer detection system in accordance with the fourth embodiment. The cancer detection system 101 differs from the system 100 in that the system 101 uses a probe 90 instead of the probe 24.

FIG. 13 shows an example of the detailed configuration of the light generator 12 also. In this example, the light generator 12 has a light source 34a, lens 34b, optical filter 34c, and lens 34d. The light source 34a emits light with a sufficiently broad wavelength range, and the lens 34b condenses and sends the light to the optical filter 34c. The optical filter 34c flattens the spectrum of the light from the light source 34a and sends the light to the lens 34d, and the lens 34d condenses the light. The light emitted from the light generator 12 in this way is the examination light.

The probe 90 has an irradiating fibre 91, a collecting fibre 92, and a fastener 93 for holding the tips of these fibres together. The irradiating fibre 91 has an end portion 91a in which optical fibres 94 are bundled, and a sheet-shaped end portion 91b in which the optical fibers 94 are arranged in a plane. On the other hand, the collecting fiber 92 is a sheet-shaped fibre in which optical fibres 95 are arranged in a plane. The sheet-shaped end portion 92b of the collecting fibre 92 is fastened to the end portion 91b of the irradiating fibre 91 by the fastener 93. The end portion 98 of the probe 90 to which the fastener is attached faces the bottom face 47 of the FOP 22. Between the end face included in the end portion 98 of the probe 90 and the bottom face 47 of the FOP 22, immersion liquid such as immersion oil may be provided. The end portion 91a receives the examination light from the light generator 34.

The end portion 91a forms a fibre bundle having the cross sectional shape corresponding to the cross sectional shape of the examination light, such as circle. The examination light received at the end portion 91a is propagated to the sheet-shaped end portion 91b by the irradiating fibre 91. As a result, the examination light having the cross section elongated in the width direction of the end portion 91b is emitted from the end portion 91b, and irradiated to the bottom face 47 of the FOP 22. Then, the examination light is transmitted through the FOP 22, and irradiated to the tissue 20 placed on the top face 46 of the FOP 22. The collecting fibre 92 receives the examination light scattered by the tissue 20 at the end portion 92b through the FOP 22. The collecting fibre 92 transmits the examination light while maintaining the one-dimensional position information along the width of the fiber 92. The examination light emerges from the sheet-shaped end portion 92a to enter the spectrometer 36. The lateral separation of the fibres at the end of the collecting fibre 92 next to the FOP 22 is sufficient to minimise cross talk between the signals collected by each fibre.

FIG. 13 shows an example of the detailed configuration of the spectrometer 36. In this example, the spectrometer 36 has a lens 36a, transmissive grating 36b, prism 36c, lens 36d, and two-dimensional photodetector 36e. The examination light emerging from the end portion 92a is collimated by the lens 36a and then dispersed by the transmission grating 36b and the prism 36c in directions corresponding to the wavelengths. The dispersed beams are condensed by the lens 36d and incident on the light-receiving face of the two-dimensional photodetector 36e. The photodetector 36e forms a vertical stripe-like image of the wavelength-dispersed beams. Thus the dispersed examination beams with different wavelengths are detected together by the two-dimensional photodetector 36e, and their intensities are measured. The measured light intensities for the respective wavelengths are sent to the control circuit 38 as the spectroscopic data and processed in a way described in the first embodiment.

The spectrometer 36 may be a reflective spectrometer using a mirror and a grating or using a concave grating having a light collecting characteristic, instead of the transmission spectrometer shown in FIG. 13. Alternatively, the spectrometer 36 may be an interference spectrometer instead of a wavelength dispersion spectrometer.

To the FOP 22, a moving mechanism 96 is connected which is able to move the FOP 22 in direction 97. The direction 97 is perpendicular to the length of the end portion 91b of the irradiating fibre 91 and to the length of the end portion 92b of the collecting fibre 92. The width of each of the end portions 91b and 92b is equal to or longer than the width of the tissue 20. Therefore the whole tissue 20 can be scanned by moving the FOP 22 in the direction 97.

Thus, for the cancer detection system 101, there is no need to move the FOP 22 in two directions individually, and all the necessary spectroscopic information can be acquired only by the movement in one direction. Consequently, it is possible to rapidly carry out the cancer examination and to simplify the moving mechanism for the scanning.

### FIFTH EMBODIMENT

The cancer detection systems in the above embodiments are post-dispersing systems that irradiate tissue with the examination light having a sufficiently broad range of wavelengths, such as white light, and disperse the examination light scattered by the tissue. However, the cancer detection system according to the invention may be a pre-dispersing system that irradiates tissue with examination light previously dispersed and detects the examination light scattered by the tissue.

FIG. 14 is a schematic view showing an optical device 12a used in a pre-dispersing cancer detection system. The optical device 12a is configured by replacing the light generator 34 in the optical device 12 with a light generator 110 and by replacing the spectrometer 36 with a detector 112. The other configuration is the same as that of the optical device 12. The pre-dispersing system is obtained by replacing the optical device 12 in the embodiments mentioned above with the optical device 12a.

The light generator 110 is able to emit examination light with a wavelength range chosen from a plurality of narrow wavelength ranges and can switch the wavelength range from one range to another. The control circuit 38 controls and causes the light generator 110 to switch the wavelength range. The light generator 110 may be configured of a light source for emitting light over a broad wavelength range as described above, and an element for choosing a narrower wavelength range from the broad wavelength range, such as a dispersion spectrometer, interference spectrometer or a wavelength tunable filter having a tunable transmitting wavelength band.

Alternatively, light-emitting devices such as a monochromator, a device having light sources (e.g., laser, LED, etc.) that emit light in their respective narrow wavelength ranges in turn, or a wavelength tunable light source (e.g., laser, LED, etc.) may be used as the light generator 110.

The photodetector 112 detects the dispersed examination light scattered by the tissue 20 via the collecting fibre 27 or 92 to generate an electric signal corresponding to its intensity and sends the signal to the control circuit 38. The control circuit 38 controls and causes the photodetector 112 to measure the intensity of the examination light at adequate timings synchronized with the light emission of the light generator 110 while causing the light generator 110 to switch the wavelength range in turn. Consequently, the intensity of the examination light is measured in each of the wavelength ranges, and therefore the spectral data of the scattered examination light is obtained. The control circuit 38 performs the pre-processing mentioned above for the spectral data, and sends the pre-processed data to the data analyzer 14. The photodetector 112 may be a zero-dimensional or one-dimensional sensor.

Since the FOP is disposed between the tissue and probe also in the post-dispersing cancer detection system, the same effects can be obtained as in the above embodiments.

### SIXTH EMBODIMENT

The cancer detection system according to the invention may be a double-dispersion system that combines the post-dispersion with the pre-dispersion. FIG. 15 is a schematic view showing an optical device 12b used in a double-dispersion cancer examination system. The optical device 12b is configured by replacing the light generator 34 in the optical device 12 with the light generator 110 mentioned above. The other configuration is the same as that of the optical device 12. The double-dispersion system is obtained by replacing the optical device 12 in the embodiments mentioned above with the optical device 12b. In this system, the tissue 20 is irradiated with the dispersed examination light from the light generator 110 and the examination light scattered by the tissue 20 is again dispersed by the spectrometer 36.

Since the FOP is disposed between the tissue and probe also in the double-dispersion cancer examination system, the same effects can be obtained as in the above embodiments.

### SEVENTH EMBODIMENT

In the seventh embodiment, the computer 40 analyses the data by calculating canonical scores as described above for each of a two-dimensional array of sampling points. Then, a diagnostic criterion was used of whether at least a predetermined number of sampling points exceeded a predetermined canonical score. It was experimentally found that the best accuracy was achieve using a diagnostic criterion for a tumour being metastatic of having nine or more scattered sampling points having a canonical score above 1.0.

### EIGHTH EMBODIMENT

In this embodiment the computer 40 analyses the data by calculating canonical scores for each sampling points as described above. Then, the size of the largest cluster of sampling points each exceeding a predetermined canonical score was identified. In this context, “touching” sampling points are adjacent sampling points, including diagonally adjacent sampling points. The eighth embodiment uses a diagnostic criterion for a tumour being metastatic of having a minimum cluster size of at least four sampling points having a canonical score above the predetermined canonical score of 1.0.

### NINTH EMBODIMENT

The methods of the seventh and eighth embodiments are complementary, and can be used together. Thus, in a particularly preferred embodiment, the computer 40 analyses tumours using both the methods described above with respect to seventh and eighth embodiments, i.e. the diagnostic criterion for a tumour being metastatic is having nine or more scattered sampling points having a canonical score above 1.0 or a minimum cluster size of at least four sampling points with a canonical score of 1.0 or above. Experiments show a sensitivity of 77% and specificity of 96% using this approach.

The present invention has been described in detail on the basis of the embodiments thereof. However, the present invention is not limited to the above embodiments. Various modifications may be made to the present invention without departing from the gist thereof.

In the above embodiments, the probe is fixed and the FOP is moved in two dimensions together with the tissue to be examined. However, the FOP and the tissue may be fixed and the probe may be moved in two dimensions. Between the end face of the probe and the FOP, other immersion liquids may be disposed instead of the immersion oil. The immersion liquid has a refractive index at least higher than that of air, i.e., 1. More preferably, the immersion liquid has the same refractive index as at least one of the cores of the FOP 22, irradiating fibre 26 and collecting fibre 27.

In the first and second embodiments, the tissue 20 touches the bottom face 47 of the FOP 22, and the top face 46 of the FOP 22 is irradiated with the examination light from the light generator 34. However, the tissue 20 may touch the top face 46 of the FOP 22, and the bottom face 47 of the FOP 22 may be irradiated with the examination light from the light generator 34. Such a configuration can be realized by using the movable stage 89 shown in FIG. 12 instead of the movable stage 16 and by adequately positioning the probe 24 and/or lens system 80, for example.

In the third and fourth embodiments, the tissue 20 touches the top face 46 of the FOP 22, and the bottom face 47 of the FOP 22 is irradiated with the examination light from the light generator 34. However, the tissue 20 may touch the bottom face 47 of the FOP 22 and the top face 46 of the FOP 22 may be irradiated with the examination light from the light generator 34. Such a configuration can be realized by using the movable state 16 instead of the movable stage 89 or moving mechanism 96 and by adequately positioning the probe 24 and mirror system 86, or the probe 90.

The spectrometer used in the cancer detection system of the invention may be one of dispersion type or interference type. The dispersion spectrometer includes, in general, a dispersion element (e.g., grating, prism etc.) and a one-dimensional or two-dimensional photoelectric converter. Examples of the dispersion spectrometer using a two-dimensional photoelectric converter are non-aberration spectrometers, which include those of reflection type and transmission type. Micro spectrometers, which are manufactured by a LIGA process, are thin, and therefore multi-dispersion can be carried out by arranging the micro spectrometers. Thus the micro spectrometers can be used similarly to non-aberration spectrometers. Multi-channel Fourier-transform spectrometers using Savart plates or the like have no movable portion, and these spectrometers may be used similarly to non-aberration spectrometers.

As the spectrometer, a plurality of wavelength selective optical filters having different transmission wavelengths may be used. An example of the wavelength selective filter is an interference filter, colour filter, or the like. For example, a filter mover may be provided which exchanges one of the wavelength selective filters disposed on the optical path for the examination light with one of the other filters. It is possible to carry out the spectroscopy by use of optical elements such as prisms (e.g., 3-prisms or 4-prisms) for dividing the optical axis of the examination light into a plurality of axes or by use of the combination of mirrors, instead of the filter mover. Wavelength selective filters may be printed on the input face of the photodetector for detecting the filtered examination light, by use of colour resist application techniques in semiconductor manufacturing. Alternatively, wavelength selective filters with photonic crystals may be used.

In the above embodiments, the tissue 20 is removed from a living body, such as a sentinel lymph node from a patient with breast cancer. However, the cancer detection system in accordance with the invention may directly examine desired portions of a living body without removing these portions from the body. Thus it is possible to determine the presence or absence of cancer other than breast cancer. For example, scanning the skin of a living body via an FOP makes it possible to detect skin cancer. Also it is possible to examine organs and tissues exposed during a surgical procedure in a living patient or removed by surgery and examined away the body.

From the invention thus described, it will be obvious that the embodiments of the invention may be varied in many ways. Such variations are not to be regarded as a departure from the scope of the invention, and all such modifications as would be obvious to one skilled in the art are intended for inclusion within the scope of the following claims.

The same cancer detection system may be used with other optical measurements for detecting cancer, including, but not limited to, fluorescence and Raman spectroscopy.

According to the present invention, it is possible to provide a cancer detection system capable of accurately and rapidly determining if cancer exists in tissue.

1. A cancer detection system, comprising:
a light generator for emitting examination light;
an irradiating fiber for propagating the examination light emitted from the light generator to irradiate tissue with the examination light;
a collecting fiber for receiving and propagating the examination light scattered by the tissue; and a
spectrum detector for detecting and recording the scattered examination light from the second light guide to measure the spectrum of the detected light;
a fibre optic plate having a first end face which transmits light entering one of the end faces to the other, with the two dimensional position of the light being maintained;
the irradiating fiber and the collecting fiber being arranged on the second end face of the light guide plate so that the light guide plate receives the examination light from the irradiating fiber and illuminates a region of the tissue corresponding to the location of the irradiating fiber and so that the light guide plate exits the examination light scattered by the said region of the tissue into the collecting fiber, and
a moving mechanism for moving the irradiating and collecting fibers with respect to the tissue to select a plurality of distinct regions of the tissue for measurement.

2. A cancer detection system according to claim 1 wherein the fibre optic plate is configured of multiple optical fibres bundled together, which transmit light entering one of the end faces to the other, with the two dimensional position of the light being maintained.

3. A cancer detection system according to claim 1 further comprising a stage on which the tissue is to be placed, wherein the moving mechanism moves the first and second light guides with respect to the tissue by moving the stage and the fibre optic plate.

4. A cancer detection system according to claim 1, wherein each of the first and second light guides is an optical fibre having an end face opposing the second end face of the fibre optic plate, and wherein the cancer detection system further comprises a member for holding the portions of the optical fibres including the end faces opposing the second end face.

5. A cancer detection system according to claim 1, wherein an immersion liquid is disposed between the second end face of the fiber optic plate and the end portions of the first and second light guides.

6. A cancer detection system according to claim 1, further comprising a lens system for transmitting the examination light from the first light guide to optically couple the examination light with the second end face of the fibre optic plate, and for transmitting the examination light scattered by the tissue and emerging from the second end face of the fibre optic plate to optically couple the scattered examination light with the second light guide.

7. A cancer detection system according to claim 1, further comprising a mirror system for reflecting the examination light from the first light guide to optically couple the examination light with the second end face of the fibre optic plate, and for reflecting the examination light scattered by the tissue and emerging from the second end face of the fibre optic plate to optically couple the scattered examination light with the second light guide.

8. A cancer detection system according to claim 1, wherein the first light guide has a first end portion in which a plurality of optical fibres are bundled, and a sheet-shaped second end portion in which the plurality of optical fibres are arranged in a line, wherein the second light guide has sheet-shaped end portions having a plurality of optical fibres arranged in a plane, wherein, in the first light guide, the first end portion receives the examination light from the light generator, and the second end portion emits the examination light to the second end face of the fibre optic plate, and wherein, in the second light guide, one of the sheet-shaped end portions receives the scattered examination light from the second end face of the fibre optic plate, and the other of the sheet-shaped end portions transmits the scattered examination light to the spectrum detector.

9. A cancer detection system according to claim 1, wherein the light generator is arranged to generate light having a predetermined range of wavelengths as the examination light, and wherein the spectrum detector includes a photodetector for measuring the intensities of a plurality of components of the scattered examination light, the components having a plurality of wavelength ranges, each of which is narrower than the predetermined wavelength range.

10. A cancer detection system according to claim 1, wherein the light generator is arranged to generate light in a plurality of wavelength ranges, the light generator being able to switch from one wavelength range to another, wherein the spectrum detector includes a photodetector for measuring the intensity of the scattered examination light, and wherein the cancer detection system further comprises a controller for causing the light generator to switch the wavelength range of the examination light from one wavelength range to another and for causing the photodetector to measure the intensity of the examination light in each of the wavelength ranges.

11-15. (canceled)

16. A cancer detection system according to claim 1 wherein the irradiating and collecting feber are arranged at distance between the centers of the irradiating and collecting fiber of 130 μm to 1000 μm apart at the second end face of the light guide plate.
